(19) United States
(12) Patent Application Publication
(10) Pub. No.: US 2025/0107806 A1
(43) Pub. Date: Apr. 3, 2025

(54) Medical device and method for safely closing, isolating, or adjusting the volume of a structure in the human body

(71) Applicants: Basilios Eleftherios SIDERIS, La Jolla, CA (US); Eleftherios Basilios SIDERIS, La Jolla, CA (US)

(72) Inventors: Eleftherios B. Sideris, Amarillo, TX (US); Basilios Eleftherios Sideris, Amarillo, TX (US)

(21) Appl. No.: 18/293,596

(22) PCT Filed: Aug. 3, 2022
(86) PCT No.: PCT/US2022/039298

§ 371 Date: Jan. 30, 2024

(30) Foreign Application Priority Data
Aug. 3, 2022 (WO) PCT/US2022/039298

(51) Int. Cl. A61B 17/12; A61B 17/00; A61B 90/00

(52) U.S. Cl. CPC A61B 17/12136; A61B 90/06; A61B 2017/00004; A61B 2017/00022; A61B 2017/00557; A61B 2017/00734; A61B 2017/00862; A61B 2017/00884; A61B 2017/1205; A61B 2090/064

(57) ABSTRACT

Disclosed is a device comprised of an occluding segment and a delivery segment whereby: the occluding segment contains one or more inflatable members; the occluding segment contains at least one component that enables controlled deflation of at least one inflatable member following permanent implantation of the device; the delivery segment contains at least one lumen to accommodate inflation of at least one inflatable member; and, the delivery segment is detachably coupled to the occluding segment on at least one point.

### CROSS-REFERENCE TO RELATED APPLICATIONS

This application is the U.S. National Stage of PCT/US2022/039298 (filed Aug. 3, 2022). PCT/US2022/039298 claims the priority and benefit of U.S. priority to U.S. Prov. Pat. App. Ser. Nos. 63/228,715 and 63/228,610, both filed Aug. 3, 2021.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### REFERENCE TO AN APPENDIX SUBMITED ON A COMPACT DISC AND INCORPORATED BY REFERENCE OF THE MATERIAL ON THE COMPACT DISC

Not applicable.

### STATEMENT REGARDING PRIOR DISCLOSURES BY THE INVENTOR OR A JOINT INVENTOR

Reserved for a later date, if necessary.

### BACKGROUND OF THE INVENION

### Field of Invention

The disclosed subject matter is in the field of medical devices intended for closure, isolation, or volumetric adjustment of physiological structures in the human body. More specifically, this document discloses significant improvements in minimally invasive devices and methods for closure, isolation or volumetric adjustment of physiological structures that adversely affect the proper flow of fluids.

### Background of the Invention

In the presence of certain physiological structures or abnormalities, fluid flow can

become modified from its normal flow patterns, potentially resulting in serious risks to patient health. Such structures can occur in a multitude of physiological systems or locations including, but not limited to the circulatory, cerebrospinal, urinary, digestive, etc. Pathogenic physiological structures can occur naturally or can be created through external causes such as trauma or surgery. In certain instances, such structures may be non-pathogenic under normal conditions, but may become pathogenic in the presence of other diseases or conditions.

In the cardiovascular system, some examples of naturally occurring pathogenic structures are congenital heart defects including Atrial Septal Defect (ASD), Ventricular Septal Defect (VSD), and Patent Ductus Arteriosus (PDA). Examples of naturally occurring structures that can become pathogenic under certain conditions include the Patent Foramen Ovale (PFO) and Left Atrial Appendage (LAA). Congenital heart defects including ASD, VSD, and PDA cause changes of cardiovascular pressures and oxygen saturation. The LAA can become the primary location of blood clot formation in patients with Atrial Fibrillation and the PFO can become the path by which blood clots from the venous circulation can cross into the arterial circulation and travel to the brain. Furthermore, certain medical procedures can create residual structures, either unintentionally or deliberately, which can become pathogenic. Examples of these include residual tunnels surrounding prosthetic valves, openings left by access sheaths in transcatheter procedures, and Arteriovenous fistulas created in dialysis patients. Although the circulatory system is the most common location where such pathogenic structures are found, they can also exist in other systems. In general terms, this invention applies to the closure, isolation, or volumetric adjustment of physiological structures that are causing abnormal or undesirable fluid flow.

A common treatment option for such structures is occlusion using a surgically or percutaneously implanted device. Devices used for this purpose generally include frames which are relatively rigid compared to the surrounding tissue. These frames are often composed of metallic materials that are intended to provide support indefinitely and maintain the device in place. Despite being successful in many ways, concerns related to persisting rigid frames remain. These may include wire fractures or interference with sensitive proximate structures such as heart valves or nodes of the cardiac conduction system. Additionally, in cases where the relative motion of the device is different to that of adjacent structures, tissue erosions can occur. Furthermore, when exposed to rapidly flowing blood, rigid materials produce high shear stresses that are associated with higher rates of thrombogenicity and reduced endothelialization. Another drawback of devices that are comprised of relatively rigid materials is that they require several sizes to safely cover the morphological range of the targeted physiological structure. Appropriate device sizing necessitates accurate measurement of the targeted physiological structure and safe implantation may require advanced image guidance, increasing the cost and complexity of a procedure. Finally, many commonly used closure devices employ the use of hooks or barbs to accomplish secure attachment to surrounding tissue. Such features can potentially be traumatic and can lead to adverse events such as pericardial effusions and can cause additional damage to surrounding tissues in cases of dislodgement or embolization.

A closure device that is based on a balloon would improve on many of the limitations of devices that rely on metallic supporting structures. A balloon-based closure device would be self-centering relative to the structure intended for closure and one or two device sizes would accommodate most physiological morphologies. Alternatively, a balloon-deliverable or otherwise expandable closure device that incorporates controllable adhesive properties would avoid the need for a supporting structure and/or hooks. Such devices would thus require fewer measurements and less advanced image guidance for appropriate implantation. Furthermore, these devices would have limited overhang onto adjacent tissues, reducing the risk of interference with sensitive neighboring structures. A balloon-based closure device would primarily rely on distention of the surrounding tissue and maximizing the contacted surface area to achieve secure apposition. Following implantation, controlled (automatic or at will) deflation of said balloon would enable the device to accommodate a desired volumetric change. A balloon-deliverable or otherwise expandable closure device would primarily rely on controlled activation of its adhesive properties both in terms of timing and location in order to enable smooth device manipulation during the procedure and secure apposition to the surrounding tissue. Additionally, for blood contacting applications, a feature that promotes endothelialization or otherwise reduces thrombogenicity would significantly improve device safety in situations where systemic antiplatelet or anticoagulant therapies are unsuitable or risky.

### SUMMARY OF THE INVENTION

In view of the foregoing, an object of this specification is to disclose novel technical embodiments of the improvements discussed in the ‘background of the invention’ section. Devices envisioned by these embodiments generally contain the following segments: occluding segment, delivery segment, and potentially or optionally an activation segment.

In a preferred embodiment, the occluding segment may be comprised of one or more independently inflatable members which are detachably or permanently attached to a distal portion of the delivery segment. The inflatable members may contain one or more valve(s), as well as a component that enables controlled deflation to take place automatically or at will post-operatively. Furthermore, the outer surface of the inflatable members may incorporate a coating designed to accomplish one or more of the following: stimulate rapid endothelialization, reduce thrombogenicity, accelerate permanent attachment to the surrounding tissue. The inflatable members may be surrounded by a separate material designed to accomplish one or more of the following: stimulate rapid endothelialization, reduce thrombogenicity, accelerate permanent attachment to the surrounding tissue. The inflatable members may contain a stem-like component, which constrains expansion in one or more directions. Optionally, the occluding segment may be formed to accommodate a wire from its proximal end to its distal end. Alternatively, the occluding segment may take the form of a patch that can be expanded to conform to the morphology of the targeted physiological opening. The patch may be impregnated with an adhesive in an inactive form. Furthermore, the patch may be designed to stimulate rapid endothelialization and/or reduce thrombogenicity. Some or all of the materials used in the implantable portions of the devices envisioned by this invention may be biodegradable/bioabsorbable under normal physiological conditions.

Preferably, the delivery segment may be comprised of one or more components that enable some or all of the following: inflation and/or deflation of the occluding segment, ‘over the wire’ advancement, detachment of a portion of the device, pressure sensing, and steerability. Alternatively, the delivery segment may be comprised of one or more independently inflatable or expandable members which are attached to the distal tip of a catheter assembly. The delivery segment may also contain components such as ports, handles, and other mechanisms which enable safe and effective implantation of the device.

The optional activation segment may suitably be comprised of at least the following: a power source, a user interface, an electronic circuit, and a transmitter/transducer. The activation segment may be used to wirelessly activate a component on the occluding portion of the device.

### BRIEF DESCRIPTION OF THE SEVERAL VIEWS OF THE DRAWINGS

Other objectives of the disclosure will become apparent to those skilled in the art once the invention has been shown and described. The manner in which these objectives and other desirable characteristics can be obtained is explained in the following description and attached figures in which:

FIG. 1 is a plan view of a preferred embodiment of a device or system comprising the segments described above in the disclosure;

FIG. 2 is a plan view of a preferred embodiment of another device or system comprising the segments described above in the disclosure;

FIG. 3 is another view of a preferred embodiment of another device or system comprising the segments described above in the disclosure;

FIG. 4 is a flow chart for another device or system comprising the segments described above in the disclosure; and,

FIG. 5 is of another device or system comprising the segments described above in the disclosure.

It is to be noted, however, that the appended figures illustrate only typical embodiments of this invention and are therefore not to be considered limiting of its scope, for the invention may admit to other equally effective embodiments that will be appreciated by those reasonably skilled in the relevant arts. Also, figures are not necessarily made to scale but are representative. In the disclosure that follows, in the interest of clarity, not all features of actual implementations are described. It will of course be appreciated that in the development of any such actual implementation, as in any such project, numerous engineering and technical decisions must be made to achieve the developers' specific goals and subgoals (e.g., compliance with system and technical constraints), which will vary from one implementation to another. Moreover, attention will necessarily be paid to proper medical and engineering practices for the environment in question. It will be appreciated that such development efforts might be complex and time-consuming, outside the knowledge base of typical laymen, but would nevertheless be a routine undertaking for those of ordinary skill in the relevant fields

### DETAILED DESCRIPTION OF PREFFERED EMBODIMENTS

Disclosed are minimally invasive devices and methods for closure, isolation or volumetric adjustment of physiological structures that adversely affect the proper flow of fluids. Such devices generally comprise: an occluding segment, a delivery segment, or an activation segment. The more specific details of such devices are disclosed with reference to the attached figures.

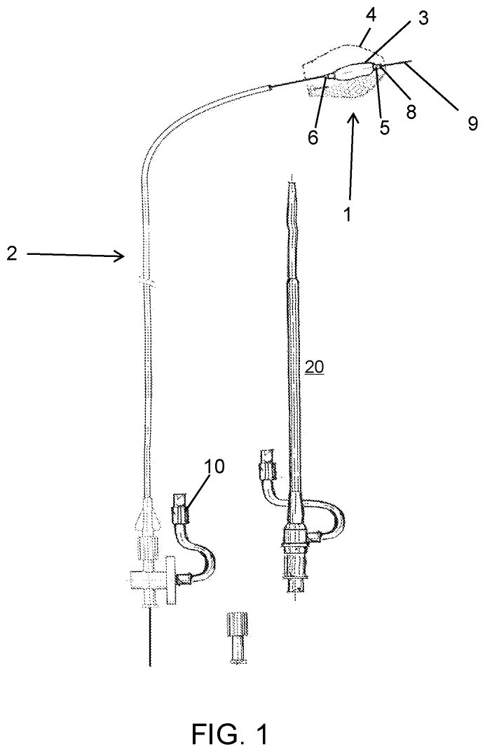

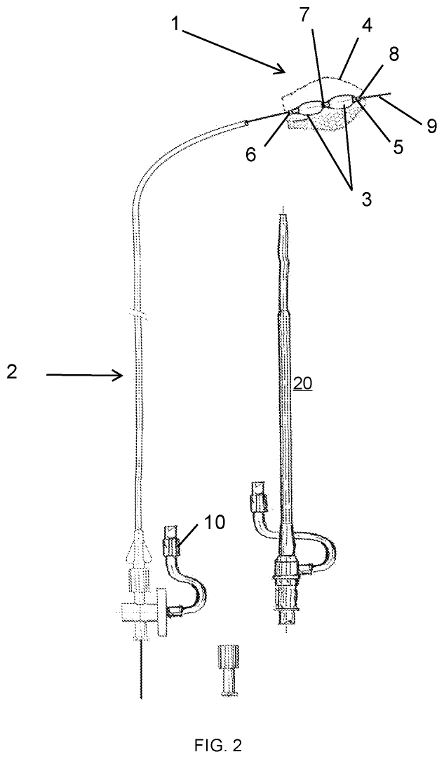

FIGS. 1 and 2 depict one embodiment of a minimally invasive device for closure, isolation or volumetric adjustment of physiological structures that adversely affect the proper flow of fluids. As shown, the device preferably includes an occluding segment (1) and a delivery segment (2). Still referring to the figures, the occluding segment (1) may be comprised of one or more inflatable member(s) (3), which are detachably coupled to the delivery segment (2). Additionally, a patch (4) may be attached to one or more points on the inflatable member(s) (3). In one version, said patch (4) may be formed by a porous and elastic material such as a polyurethane foam.

Still referring to FIGS. 1 and 2, the occluding segment (1) may include one or more valves (6) in order to maintain a pressure differential between different inflatable members and/or with the external environment. The occluding segment (1) may also include one or more controlled deflation component(s) (5) that can deflate the inflatable member(s) (3) automatically or at will, post-operatively. In one embodiment of the occluding segment (1), each inflatable member (3) may be independently inflated/deflated through a separate lumen. In another embodiment of the occluding segment (1), multiple inflatable members (3) may be independently inflated/deflated through the same lumen.

As shown in FIG. 2, a valve (6) may be positioned at the proximal end of the most proximal inflatable member (3) and valve/gasket components (7) (FIG. 2) may be positioned between adjacent inflatable members (3). In one mode of operation, inflation/deflation of each inflatable member (3) may be achieved by crossing one or more valve/gasket components (7) (including the proximal valve) with a dedicated inflation catheter, through which fluid may either be injected or removed. The dedicated inflation catheter may optionally be capped at its end and have at least one set of holes along the length of its tip. When attempting to inflate/deflate two adjacent inflatable members (3), the tip of the inflation catheter may be placed through the joining valve/gasket component (7) and positioned in a way that exposes the holes at the tip of the catheter to one inflatable member (3) at a time. The position of the inflation catheter may then be changed to inflate the other inflatable member (3). In another embodiment of the inflatable member(s) (3), the end of an inflation catheter may be temporarily attached to the base of the proximal valve (6), allowing fluid to pass from the catheter and through the valve (6). In another embodiment of the occluding segment (1), two or more inflatable members (3) may be attached to one another without valve/gasket component(s) (7), in which case they may be inflated simultaneously through the same inflation catheter.

In any of these embodiments, a valve (8) may be positioned at the distal end of the most distal inflatable member (3) to accommodate an ‘over the wire’ procedure. Said ‘over the wire’ procedure may be performed by passing a wire (9) through the distal valve (6) and advancing it through the proximal side of the occluding segment (1) either directly through the intermittent valve/gasket components (7) and finally the proximal valve (8), or through the tip of an inflation catheter which has been positioned inside the occluding segment (1). Suitably, the inflatable member(s) (3) may be inflated with the wire (9) in place, enabling deflation and device repositioning if necessary.

In yet another embodiment of the occluding segment (1), the inflatable member(s) (3) may be rigidly attached at one or more points along a stem-like member which may include one or more lumens traversing at least a portion of the stem-like member (i.e. a lumen that is capped on one end). In cases where the inflatable member(s) (3) are compliant, the stem-like member may be made from a material with a higher elastic modulus than that of the inflatable member(s) in order to constrain expansion of the inflatable member(s) along its axis. The lumens may include one or more holes providing access to one or more inflatable members (3) and said stem-like member may be rigid or flexible. Valves may be attached directly to the stem-like member in order to achieve inflation or deflation of the inflatable member(s), and advancement of a wire through the inflatable member(s). Inflation, deflation, and advancement of a wire may be achieved through the same lumen, similarly to the embodiments which do not include a stem-like member. Alternatively, separate lumens may be used for advancement of a wire and inflation/deflation of the inflatable member(s) (3).

In any of the aforementioned embodiments of the occluding segment (1), one or more controlled deflation component(s) (5) may be positioned within or on one or more inflatable member(s). Said controlled deflation component(s) (5) may be partly or wholly coated with a suitable insulating material. In one embodiment of said controlled deflation component(s) (5), the component(s) (5) may contain a barrier made from a biodegradable material that has been designed to degrade within a specific timeframe under normal physiological conditions, thus causing deflation. In another embodiment, said controlled deflation component(s) (5) may incorporate the following: an actuator, a sensor, a control circuit, and a local power source (e.g. lithium ion cell, supercapacitor, thermochemical reaction, etc.). In said embodiment, when the sensor detects a condition corresponding to a predetermined state, it would activate the control circuit and in turn, the actuator.

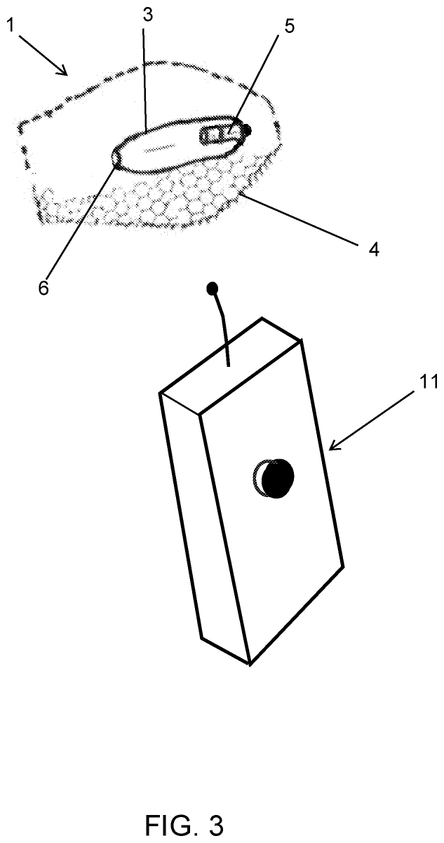

In another embodiment, as seen in FIG. 3, said controlled deflation component(s) (5) may incorporate the following: an actuator, a receiver, a control circuit, and a local power source (e.g. lithium ion cell, supercapacitor, thermochemical reaction, etc.). In said embodiment, a user would remotely activate the actuator. In another embodiment, said controlled deflation component(s) (5) may be powered by a local power receiver, coupled to a remote power source (e.g. ultrasonic transducer, electromagnetic transmitter, etc.). In said embodiment, the actuator may be coupled directly to a local power receiver, obviating the need for a control circuit. Alternatively, the actuator may itself be the local power receiver. In any of the aforementioned embodiments, the physical deformation of the actuator may be driven electrically, chemically, thermally, mechanically or by any combination thereof. In yet another embodiment of the controlled deflation component (5), a valve mechanism may be designed in a way that leverages the deployed configuration of the occluding segment and/or the surrounding tissue, in order to establish a threshold of mechanical forces across which a state change (open/closed) takes place.

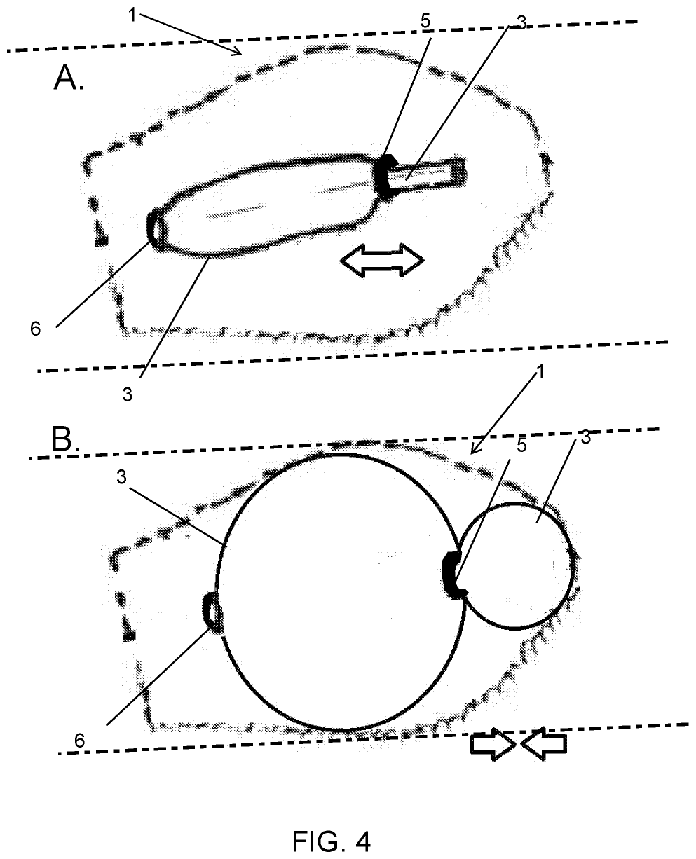

One version of this embodiment may include a valve, positioned between two inflatable segments, as seen in FIG. 4. When both segments are inflated within a pouch-like structure, compression forces form between the two inflatable members, closing the valve. If the occluding segment becomes dislodged in any way, the compression forces between the two members would dissipate, causing the valve to open. Additionally, in any of the aforementioned embodiments, the occluding segment may include a feature which allows it to be detachably coupled to the delivery segment. This feature may include one or more of the following: lumen, threaded lumen, barb, gap, groove, hole, thread loop. These parts may be used in conjunction with a mechanism on the distal part of the delivery segment that enables the occluding segment to be altered between two configurations, attached and detached. One embodiment of said detachment mechanism may include a wire with a threaded tip which passes through the delivery segment and screws into a threaded lumen on the occluding segment. In another embodiment, the detachment mechanism may include a non-threaded wire which passes through the delivery segment and into a non-threaded lumen in/on the occluding segment. The non-threaded lumen in/on the occluding segment may contain a small gap that exposes it to the external environment. In the attached configuration, a thread that is attached to the occluding segment would be looped around the wire through the small gap, thus locking the occluding and delivery segments together. Pulling the wire would release the thread and in that way, detach the occluding segment. In a third embodiment, a thread may be passed through the delivery segment and tightened around a bulb, gap, groove, or thread loop on the occluding segment. In order to achieve detachment, the thread may be loosened and/or removed. In yet another embodiment, a thread loop, attached to and extending from the occluding segment, may be looped around a partially exposed wire which extends across two lumens in the delivery segment. In order to achieve detachment, the wire may be partially or entirely removed. All aforementioned embodiments may also serve to maintain proper alignment between the occluding and delivery segments.

The delivery segment may include one or more independent or connected lumens which may be aligned with one or more components (e.g. lumen, valve, etc.) in the occluding segment. At least one lumen may be used for inflation or deflation of the balloon member. Such a lumen may accommodate a catheter which may be independently advanced to or retracted from the occluding member. In the advanced position, where the tip of the catheter has crossed one or more valve/gasket components in the occluding segment, both inflation and deflation may be achieved by injecting or removing fluid through the catheter. Such a lumen may also accommodate a wire or thread. Alternatively, a dedicated lumen may be used to accommodate a wire or thread. The curvature of the delivery segment may be set in a way that enables smooth advancement to the target physiological structure. For example, an L shaped distal end may facilitate advancement to the Left Atrial Appendage and a J shaped distal end may facilitate advancement to the Ventricular Septum. A steerable component may be added to the delivery segment, either within one or more lumens as a steerable wire or surrounding one or more lumens as a steerable catheter. The proximal end of the delivery segment may include ergonomic controls such as push, pull, or rotary mechanisms to deliver, detach, or retrieve the occluding segment. Additionally, the proximal end of the delivery segment may include one or more ports for inflation/deflation of the inflatable member(s), as well as controls to steer the device into place. One such port may be connected to a pressure sensor (10) in order to facilitate measurement of the balloon pressure throughout the implantation procedure. Any components of the delivery segment that provide for a conduit between a physiological system and the external environment may incorporate valves or gaskets in order to prevent air embolism.

Referring back to FIG. 3, an optional and separate activation segment (11) may be used to achieve deflation of the occluding segment post-operatively. Said activation segment (11) may be comprised of at least the following: a power source, a user interface, an electronic circuit, and a transmitter/transducer. The activation segment (11) may be used to wirelessly activate one or more component(s) on the occluding segment of the device in order to cause deflation. In one embodiment, the activation segment (11) may contain hardware and software required to transmit a suitable activation signal to the deflation component(s). The activation signal may be encoded or unencoded and in certain embodiments of the invention, may be used to power the activation component(s) directly.

Implantation of the aforementioned embodiments of this device may be carried out according to the following steps. Access to the physiological system of interest may be gained using commonly used surgical or percutaneous methods. A wire may be advanced to the physiological structure targeted for closure, isolation, or volumetric adjustment. A short or long delivery sheath (20), which is able to accommodate the device described in this invention, may be advanced ‘over the wire’ into the physiological system. The portion of the wire that extends outside of the patient may then be passed through the distal tip of the device and advanced through to its proximal end. The device may then be advanced ‘over the wire’, through the sheath, and to the physiological structure targeted for closure, isolation or volumetric adjustment. Alternatively, the wire may be withdrawn following placement of the sheath and the device may be advance through the sheath independently. If available, steerability may be used during device advancement, as necessary. The device may then be inflated and detached. In an ‘over the wire’ procedure, withdrawal of the wire may be performed at any time prior to detachment of the occluding segment. When retracting components through the occluding segment, during or after detachment from the delivery segment, an additional counter force may be provided by positioning the tip of the delivery segment on the proximal end of the occluding segment. Finally, the delivery sheath may be retracted and the percutaneous entry point may be sutured. If the external activation segment is present and deflation becomes desirable at any point following detachment of the occluding segment, the deflation component(s) may be activated. Retrieval of the occluding segment may then be performed using known surgical or percutaneous methods.

The present invention also encompasses devices and methods for safely implanting, attaching, and releasing an occlusive patch placed at the site of a physiological opening. The implantation procedure of one such occlusive device is described in detail in our previous U.S. Pat. No. 6,238,416 (previously incorporated by reference herein) and as such will only be described herein to the extent that it is necessary to illustrate the novel aspects of the present invention.

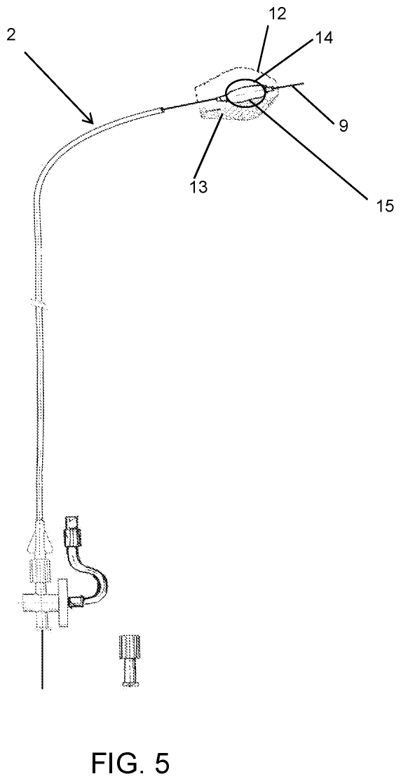

In accordance with one embodiment of the invention, the device includes an occluding segment (1) and a delivery segment (2), as seen in FIG. 5. The occluding segment (1) may take the form of a patch (12) that can be expanded to conform to the morphology of the targeted physiological opening. The patch (12) may be impregnated with an adhesive (13) in an inactive form either as part of the manufacturing process, directly before implantation, or during implantation. Said adhesive may be polyethylene glycol based with end/side groups that can react with each other and the surrounding tissue to form strong chemical bonds. More generally, the adhesive may take the form of a liquid, gel, or solid and may be chemically or mechanically bound to the patch. In one embodiment, the adhesive may be placed on the entire patch or on a selected portion of the patch prior to implantation. Once the device is positioned correctly in the appropriate physiological opening, the adhesive may be activated by ejecting a biocompatible fluid to certain parts of the patch. Said fluid, may be a chemical initiator that activates the adhesive directly or it may be used to change a feature in the macroenvironment surrounding the patch (e.g. temperature, pH, etc.) which in turn activates the adhesive. In another embodiment, the adhesive may be ejected directly onto a portion or the entirety of the patch, either in an active or inactive form.

The delivery segment (2) may include one or more independent or connected lumens which may be aligned with one or more components in the occluding segment. At least one lumen may be used for inflation/expansion or deflation/contraction of a distal member. In one embodiment, said distal member may be comprised of a double-walled balloon with a porous walled outer balloon (14) disposed over an inner balloon (15) (which may be porous or non-porous). Within the delivery segment, a first lumen may communicate from the proximal end to the inner balloon, and a second lumen may communicate from the proximal end to the space between the inner balloon and outer balloon. The porous outer balloon may comprise standard balloon materials such as nylons, block co-polymers (PEBAX), urethanes, PET, PE (HMWPE, LLDPE, etc.), with numerous sub millimeter pores and may be compliant (elastomeric and conformable to the target physiological structure) or non-compliant, while the inner balloon may be non-porous or porous, and also may be elastomeric and conformable to the target physiological structure (or outer balloon) or non-compliant. In one embodiment of the delivery segment, the adhesive may be located in a solid and/or inactive form in the space between the inner and outer balloons.

Implantation of the aforementioned embodiments of this invention may be carried out according to the following steps. Access to the physiological system of interest may be gained using commonly used surgical or percutaneous methods. A wire may be advanced to the physiological structure targeted for closure, isolation, or volumetric adjustment. A short or long sheath, which is able to accommodate the device described in this invention, may be advanced ‘over the wire’ into the physiological system. At this point, if not already present, the adhesive may be applied directly to the patch by the operator. The portion of the wire that extends outside of the patient may then be passed through the distal tip of the device and advanced through to its proximal end. The device may then be advanced ‘over the wire’, through the sheath, and to the physiological structure targeted for closure, isolation or volumetric adjustment. Alternatively, the wire may be withdrawn following placement of the sheath and the device may be advance through the sheath independently. If available, steerability may be used during device advancement, as necessary. The inflatable portions of the device may then be inflated. In one embodiment, the inner balloon may be inflated first in order to position the device, followed by inflation of the outer balloon to achieve controlled ejection of the activating fluid and/or adhesive. In another embodiment, the outer balloon may be partly inflated (loaded), followed by inflation of the inner balloon to force ejection from the outer balloon. Following a pre-specified waiting period, the balloon catheter may then be deflated and removed. Secure apposition of the patch may then be confirmed, and the patch may be release. Finally, the delivery sheath may be retracted, and the percutaneous entry point may be sutured.

Although the method and apparatus is described above in terms of various exemplary embodiments and implementations, it should be understood that the various features, aspects and functionality described in one or more of the individual embodiments are not limited in their applicability to the particular embodiment with which they are described, but instead might be applied, alone or in various combinations, to one or more of the other embodiments of the disclosed method and apparatus, whether or not such embodiments are described and whether or not such features are presented as being a part of a described embodiment. Thus the breadth and scope of the claimed invention should not be limited by any of the above-described embodiments.

Terms and phrases used in this document, and variations thereof, unless otherwise expressly stated, should be construed as open-ended as opposed to limiting. As examples of the foregoing: the term “including” should be read as meaning “including, without limitation” or the like, the term “example” is used to provide exemplary instances of the item in discussion, not an exhaustive or limiting list thereof, the terms “a” or “an” should be read as meaning “at least one,” “one or more,” or the like, and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that might be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases might be absent. The use of the term “assembly” does not imply that the components or functionality described or claimed as part of the module are all configured in a common package. Indeed, any or all of the various components of a module, whether control logic or other components, might be combined in a single package or separately maintained and might further be distributed across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives might be implemented without confinement to the illustrated examples. For example, block diagrams and their accompanying description should not be construed as mandating a particular architecture or configuration.

All original claims submitted with this specification are incorporated by reference in their entirety as if fully set forth herein.

I claim:

1. A device comprising:
an occluding segment, wherein the occluding segment contains one or more inflatable members, the occluding segment contains at least one component that enables controlled deflation of at least one inflatable member following permanent implantation of the device; and,
a delivery segment wherein the delivery segment contains at least one lumen to accommodate inflation of at least one inflatable member and the delivery segment is detachably coupled to the occluding segment on at least one point.

2. The device from claim 1 wherein at least one inflatable member contains at least one valve.

3. The device from claim 2 wherein the at least one inflatable member is attached to at least one stem-like member on at least one point.

4. The device from claim 2 whereby each inflatable member can be inflated/deflated independently.

5. The device from claim 1 wherein the inflatable members are surrounded by a material intended to achieve a result selected from a group consisting essentially of the following: accelerated permanent attachment to the surrounding tissue;
accelerated endothelialization; or reduced thrombogenicity.

6. The device from claim 5 wherein the material forms a patch, wherein the patch is attached to at least one inflatable member on at least one point, the patch is elastic; and the patch is porours.

7. The device from claim 1 wherein the occluding segment accommodates a guide wire for ‘over the wire’ implantation.

8. The device from claim 1 wherein the component for controlled deflation takes a form selected from the group consisting essentially of:
a. a biodegradable barrier;
b. an actuation system that incorporates at least one of the following features:
i. an actuator;
ii. a receiver;
iii. a transmitter/transducer;
iv. an electronic circuit;
v. a power source;
vi. a sensor;
vii. a user interface; or
c. a valve mechanism that changes state based on crossing a threshold of mechanical forces set by the deployed configuration of at least one inflatable member of the occluding segment.

9. The device from claim 1 wherein the component for controlled deflation is coated by an insulating material.

10. The device from claim 1 wherein the occluding segment and delivery segment maintain alignment throughout deployment.

11. The device from claim 1 wherein the delivery segment incorporates a steerable mechanism.

12. The device from claim 1 wherein the delivery segment incorporates at least one pressure sensor.

13. The device from claim 1 whereby the component for controlled deflation can be activated by an external controller.

14. A method of placing a device at the site of a physiological structure comprising:
a. placing into a delivery sheath an occluding segment that is detachably coupled to a delivery segment, wherein said occluding segment contains at least one inflatable member and at least one component that enables controlled deflation of the at least one inflatable member following permanent implantation of the device
b. advancing the occluding segment through the delivery sheath to the physiological structure of interest via the delivery segment;
c. inflating the at least one inflatable member of the occluding segment;
d. detaching the occluding segment from the delivery segment; and
e. deflating the at least one member.
